(19) United States
(12) Patent Application Publication
(10) Pub. No.: US 2022/0324882 A1
(43) Pub. Date: Oct. 13, 2022

(54) FUNCTIONALIZED METALS, SYNTHESES THEREOF, AND USES THEREOF

(71) Applicant: Honda Motor Co., Ltd., Tokyo (JP)

(72) Inventors: Shutang Chen, Livermore, CA (US); Gugang Chen, Palo Alto, CA (US)

(21) Appl. No.: 17/227,114

(22) Filed: Apr. 9, 2021

(51) Int. Cl. C07F 1/00; G01N 27/04

(52) U.S. Cl. CPC C07F 1/005; G01N 27/04; B82Y 40/00

(57) ABSTRACT

Aspects of the present disclosure generally relate to functionalized metals, to processes for producing functionalized metals, and to uses of functionalized metals as, e.g., sensing materials for chemiresistive sensors. In an aspect, a process for producing a functionalized metal is provided. The process includes introducing, under first conditions, a first precursor comprising a Group 10 to Group 14 metal with an amine to form a second precursor comprising the Group 10 to Group 14 metal. The process further includes introducing, under second conditions, the second precursor with a third precursor to form the functionalized metal, the third precursor comprising an organic material having the formula HS—R—COOH, wherein R is an unsubstituted hydrocarbyl, a substituted hydrocarbyl, an unsubstituted alkoxy, or a substituted alkoxy.

### FIELD

Aspects of the present disclosure generally relate to functionalized metals, to processes for producing functionalized metals, and to uses of functionalized metals as, e.g., sensing materials for chemiresistive sensors.

### BACKGROUND

Chemiresistive sensors change their electrical resistance in response to variations in the chemical environment based on chemical interactions between the sensing material of the sensor and the analyte. Because the sensing material has an inherent resistance, interactions (e.g., bonding or absorption) of the analyte to the sensing material restricts the flow of electrons in the sensing material, causing the resistance of the sensing material to change. Chemiresistive sensors to detect various analytes, such as ammonia (NH₃), find use in a variety of applications. For example, such sensors are used in industrial situations where gases pose a risk to worker safety, automotive applications to detect gas emissions, as well as environmental gas analysis where field-portable monitors based on chemiresistive sensors enable measuring and monitoring of trace amounts of gases.

Conventional sensing materials for chemiresistive sensors include metal oxides, conducting polymers, or carbon nanostructures as the sensing material. Such sensing materials, however, suffer from various drawbacks. For example, conducting polymers are thermally unstable and cannot be used at temperatures where gas-solid interactions proceed rapidly. As another example, metal oxides such as SnO₂ require high operating temperatures for analyte selectivity. Carbon nanostructures lack sensitivity, rendering them inoperable at low concentration levels of gaseous species. Moreover, the fabrication of nanostructures can be complex. Besides these drawbacks of analyte selectivity, sensitivity, and long-term stability issues, chemiresistive sensors made of state-of-the-art sensing materials lack portability and have poor response time.

There is a need for improved materials, processes for making such materials, and to uses of such materials as, e.g., sensing materials, that overcome one or more of the aforementioned deficiencies of conventional sensing materials.

### SUMMARY

Aspects of the present disclosure generally relate to functionalized metals, to processes for producing functionalized metals, and to uses of functionalized metals, e.g., sensing materials for chemiresistive sensors.

In an aspect, a process for producing a functionalized metal is provided. The process includes introducing, under first conditions, a first precursor comprising a Group 10 to Group 14 metal with an amine to form a second precursor comprising the Group 10 to Group 14 metal. The process further includes introducing, under second conditions, the second precursor with a third precursor to form the functionalized metal, the third precursor comprising an organic material having the formula HS—R—COOH, wherein R is an unsubstituted hydrocarbyl, a substituted hydrocarbyl, an unsubstituted alkoxy, or a substituted alkoxy.

In another aspect, a composition is provided. The composition includes a Group 10 to Group 14 metal, and an organic group bonded to the Group 10 to Group 14 metal, the organic group comprising [symbol] S—R—COOH, wherein [symbol] represents a bond to the Group 10 to Group 14 metal; and R is an unsubstituted hydrocarbyl, a substituted hydrocarbyl, an unsubstituted alkoxy, or a substituted alkoxy.

In another aspect, a device for detecting an analyte is provided. The device includes a substrate, a source electrode and a drain electrode disposed on the substrate, and a film disposed on a surface of the substrate. The film includes a Group 10 to Group 14 metal and an organic group bonded to the Group 10 to Group 14 metal, the organic group comprising [symbol] S—R—COOH, wherein [symbol] represents a bond to the Group 10 to Group 14 metal, and R is an unsubstituted hydrocarbyl, a substituted hydrocarbyl, an unsubstituted alkoxy, or a substituted alkoxy.

### BRIEF DESCRIPTION OF THE DRAWINGS

So that the manner in which the above recited features of the present disclosure can be understood in detail, a more particular description of the disclosure, briefly summarized above, may be had by reference to aspects, some of which are illustrated in the appended drawings. It is to be noted, however, that the appended drawings illustrate only exemplary aspects and are therefore not to be considered limiting of its scope, for the disclosure may admit to other equally effective aspects.

FIG. 1 is an illustration of an example functionalized metal according to at least one aspect of the present disclosure.

FIG. 2 is a flowchart showing selected operations of an example process for producing a functionalized metal according to at least one aspect of the present disclosure.

FIG. 3 shows example reaction diagrams for converting a metal alkylamine to a functionalized metal according to at least one aspect of the present disclosure.

FIG. 4A is an illustration of a side view of an example device for detecting an analyte according to at least one aspect of the present disclosure.

FIG. 4B is a pictorial representation of ammonia (NH₃) sensing with an example functionalized metal according to at least one aspect of the present disclosure.

FIG. 4C shows exemplary resistance data of an example functionalized metal bound to NH₃ according to at least one aspect of the present disclosure.

FIG. 4D shows an example device for detecting an analyte according to at least one aspect of the present disclosure.

FIG. 5 is an exemplary transmission electron microscope (TEM) image of example mercaptosuccinic acid-modified gold (Au) (MSA-modified Au) nanoparticles according to at least one aspect of the present disclosure.

FIG. 6 is an exemplary X-ray diffraction (XRD) pattern of example MSA-modified Au nanoparticles according to at least one aspect of the present disclosure.

FIG. 7 is an exemplary ultraviolet-visible (UV-Vis) absorption spectrum of example MSA-modified Au nanoparticles according to at least one aspect of the present disclosure.

FIG. 8 shows exemplary Fourier transform infrared (FT-IR) spectra for mercaptosuccinic acid and example MSA-modified Au nanoparticles according to at least one aspect of the present disclosure.

FIG. 9A is a scanning electron microscope (SEM) image of an example film according to at least one aspect of the present disclosure (Scale: 500 μm).

FIG. 9B is an SEM image of the example film of FIG. 9A (Scale: 300 nm) according to at least one aspect of the present disclosure.

FIG. 10A shows exemplary resistance data for an example sensor device, which includes example MSA-modified Au nanoparticles as the sensing material, in the presence of NH₃ and nitrogen dioxide (NO₂) according to at least one aspect of the present disclosure

FIG. 10B shows the concentrations of NH₃ and NO₂ pulsed at selected time points during measurement of the resistance data shown in FIG. 10A according to at least one aspect of the present disclosure.

FIG. 11A shows exemplary resistance data for an example sensor device, which includes example 3-mercaptopropionic acid-modified Au nanoparticles as the sensing material, in the presence of NH₃ and NO₂ according to at least one aspect of the present disclosure.

FIG. 11B shows the concentrations of NH₃ and NO₂ pulsed at selected time points during measurement of the resistance data shown in FIG. 11A according to at least one aspect of the present disclosure.

FIG. 12A shows exemplary resistance data for an example sensor device, which includes example mercaptopoly(ethylene glycol) carboxylic acid-modified Au nanoparticles as the sensing material, in the presence of NH₃ and NO₂ according to at least one aspect of the present disclosure.

FIG. 12B shows the concentrations of NH₃ and NO₂ pulsed at selected time points during measurement of the resistance data shown in FIG. 12A according to at least one aspect of the present disclosure.

FIG. 13A shows exemplary resistance data for an example sensor device, which includes example MSA-modified Au nanoparticles as the sensing material, in the presence of NH₃ according to at least one aspect of the present disclosure.

FIG. 13B shows exemplary resistance data for an example sensor device, which includes example MSA-modified silver (Ag) nanoparticles as the sensing material, in the presence of NH₃ according to at least one aspect of the present disclosure.

FIG. 13C shows exemplary resistance data for an example sensor device, which includes example MSA-modified copper (Cu) nanoparticles as the sensing material, in the presence of NH₃ according to at least one aspect of the present disclosure.

FIG. 14A shows exemplary resistance data for an example sensor device, which includes example MSA-modified Au nanoparticles as the sensing material, in the presence of nitric oxide (NO), carbon dioxide (CO₂), carbon monoxide (CO), hydrogen (H₂), ethanol, acetone, NH₃, nitrogen dioxide (NO₂), and methane (CH₄), according to at least one aspect of the present disclosure.

FIG. 14B shows the concentration of each gas pulsed at selected time points during measurement of the resistance data shown in FIG. 14A according to at least one aspect of the present disclosure.

FIG. 15 shows exemplary sensor response data for an example sensor device, which includes example MSA-modified Au nanoparticles as the sensing material, in the presence of NO, CO₂, CO, H₂, ethanol, acetone, NH₃, NO₂, and CH₄, according to at least one aspect of the present disclosure.

FIG. 16 shows exemplary resistance data for an example sensor device, which includes example MSA-modified Au nanoparticles as the sensing material, in the presence of NH₃ according to at least one aspect of the present disclosure.

To facilitate understanding, identical reference numerals have been used, where possible, to designate identical elements that are common to the figures. It is contemplated that elements and features of one example may be beneficially incorporated in other examples without further recitation.

### DETAILED DESCRIPTION

Aspects of the present disclosure generally relate to functionalized metals and to processes for producing functionalized metals. The functionalized metals can be utilized for, e.g., analyte sensing in a variety of applications including gas monitoring in industrial, transportation, and environmental settings. In these and other applications, the functionalized metals can be at least a portion of a sensing material of a sensor device (e.g., chemiresistive sensor). Briefly, the functionalized metal includes a metal bound to an organic group (also referred to as an organic functional group). The organic group includes a mercapto moiety (or thiol moiety) and a carboxyl moiety. The metal is bound to the sulfur atom of the mercapto moiety such that the carboxyl moiety is located distal to the metal. During analyte sensing, the carboxyl moiety can interact with an analyte, and a change in resistance due to the interaction is detected, monitored, measured, determined, or otherwise sensed.

With respect to ammonia sensing, conducting polymers, semiconductive metal oxides, and nanostructures have been heavily investigated as sensing materials. While conducting polymers can be utilized for room temperature detection of ammonia, their lack of long-term stability and sensitivity to humidity and air remain challenges to their implementation. While semiconductive metal oxides (e.g., SnO₂) exhibit better stability than conducting polymers, conventional sensors incorporating such metal oxides must operate at temperatures above 400° C. for high selectivity.

In contrast to conventional sensing materials, the functionalized metals described herein can operate at room temperature (e.g., from about 15° C. to about 25° C.). The functionalized metals described herein also exhibit excellent sensitivity. For example, target ammonia species can be detected at concentration levels in the parts-per-million (ppm) range and parts-per-billion (ppb) range, even in the presence of other gases such as carbon monoxide (CO), nitrogen dioxide (NO₂), and methane (CH₄).

### Functionalized Metals

The present disclosure generally relates to functionalized metals, and more specifically to carboxyl functionalized metals. With its free carboxyl group (COOH) or carboxylate ion (COO⁻), such functionalized metals can be useful for, e.g., detecting, monitoring, measuring, determining, or otherwise sensing analytes such as ammonia.

The functionalized metal can be in the form of a complex, a coordination compound, or the like. The functionalized metal can be a composition or form a portion of a composition. The functionalized metal(s) can be in the form of a structure such as particles (nanoparticles, microparticles, or macroparticles), a monolayer film, a multilayer film, or other structure as described below.

The functionalized metal includes, or is selected from, one or more elements (e.g., metals) from Group 10 to Group 14 of the periodic table of elements, such as Ni, Pd, Pt, Cu, Ag, Au, Cd, Hg, Zn, Al, Ga, In, Tl, Sn, Pb, or combinations thereof. The Group 10 to Group 14 metal is bound (chemically and/or physically) to one or more organic groups. The organic group(s) can have the formula:

[symbol]

S—R—X¹,

wherein: “ [symbol] ” represents a bond to the Group 10 to Group 14 metal;

X¹ is, or is selected from, carboxyl (COOH) or carboxylate ion (COO⁻); and

R is, or is selected from, an unsubstituted hydrocarbyl (such as a C₁-C₁₀₀ unsubstituted hydrocarbyl, such as C₁-C₄₀ unsubstituted hydrocarbyl, such as C₁-C₂₀ unsubstituted hydrocarbyl, such as C₁-C₁₀ unsubstituted hydrocarbyl, such as C₁-C₆ unsubstituted hydrocarbyl), a substituted hydrocarbyl (such as a C₁-C₁₀₀ substituted hydrocarbyl, such as C₁-C₄₀ substituted hydrocarbyl, such as C₁-C₂₀ substituted hydrocarbyl, such as C₁-C₁₀ substituted hydrocarbyl, such as C₁-C₆ substituted hydrocarbyl), an unsubstituted alkoxy (such as a C₁-C₁₀₀ unsubstituted alkoxy, such as C₁-C₄₀ unsubstituted alkoxy, such as C₁-C₂₀ unsubstituted alkoxy, such as C₁-C₁₀ unsubstituted alkoxy, such as C₁-C₆ alkoxy), a substituted alkoxy (such as a C₁-C₁₀₀ substituted alkoxy, such as C₁-C₄₀ substituted alkoxy, such as C₁-C₂₀ substituted alkoxy, such as C₁-C₁₀ substituted alkoxy, such as C₁-C₆ substituted alkoxy), an unsubstituted aryl (such as a C₄-C₁₀₀ unsubstituted aryl, such as C₄-C₄₀ unsubstituted aryl, such as C₄-C₂₀ unsubstituted aryl, such as C₄-C₁₀ unsubstituted aryl), or a substituted aryl (such as a C₄-C₁₀₀ substituted aryl, such as a C₄-C₄₀ substituted aryl, such as C₄-C₂₀ substituted aryl, such as C₄-C₁₀). R can be can be saturated or unsaturated, linear or branched, cyclic or acyclic, aromatic or non-aromatic.

In at least one aspect, and when R is a substituted hydrocarbyl, a substituted alkoxy, or a substituted aryl, at least one carbon of the substituted hydrocarbyl, the substituted alkoxy, the substituted aryl has been substituted with at least one heteroatom or heteroatom-containing group, such as one or more elements from Group 13-Group 17 of the periodic table of the elements, such as halogen (e.g., F, Cl, Br, or I), O, N, Se, Te, P, As, Sb, S, B, Si, Ge, Sn, Pb, and the like, such as NR*₂, OR* (e.g., OH or O₂H), SeR*, TeR*, PR*₂, AsR*₂, SbR*₂, SR*, SOX (where x=2 or 3), BR*₂, SiR*₃, GeR*₃, SnR*₃, PbR*₃, and the like, where each R* is independently hydrogen, a hydrocarbyl radical (e.g., C₁-C₁₀), or two or more R* may join together to form a substituted or unsubstituted, fully saturated, partially unsaturated, fully unsaturated, aromatic, cyclic, or polycyclic ring structure. “Alkoxy” includes ethers and polyethers.

R can include polymers, such as homopolymers and copolymers. Illustrative, but non-limiting, examples of polymers can include, or can be selected from, polyglycols (also called polyethers, polyether glycols or polyols), such as polyethylene glycol (PEG), polyethylene oxide (PEO), polyoxyethylene (POE), polypropylene glycol (PPG), polypropylene oxide (PPDX), polyoxypropylene (POP), polytetramethylene glycol (PTMG), polytetramethylene ether glycol (PTMEG), polytetrhadrofuran, polyacetal, and paraformaldehyde; polyaryls such as polymers having styrene, aniline; polyolefins such as polyethylene or polypropylene; polyesters such as polyethylene terepthalate (PET); polyureas and block copolymers thereof such as polyurethaneureas; polyurethanes, including polyurethane block copolymers; polyethers, including polyether copolymers such as polyether-polyurea copolymers; polyparaphenylenevinylenes, polyanilines, polyazines, polythiophenes, poly-p-phenylene sulfides, polyfuranes, polypyrroles, polyselenophene, and/or polyacetylenes. The average number molecular weight (Mₙ) of the polymers can be from about 250 g/mol to about 100,000 g/mol, such as from about 450 g/mol to about 50,000 g/mol, such as from about 650 g/mol to about 25,000 g/mol, such as from about 900 g/mol to about 10,000, such as from about 2,000 g/mol to about 7,500, such as from about 3,000 g/mol to about 6,000 g/mol, such as from about 4,000 to about 5,000 g/mol.

In at least one aspect, the organic group has the formula:

HS—CₓH_{y}—(COOH)_{z},

wherein:
x is a positive number such as about 1 or more, such as from about 1 to about 5,000, such as from about 1 to about 500, such as from about 1 to about 50, such as from about 1 to about 10;
y is a positive number such as about 1 or more, such as from about 1 to about 10,000, such as from about 1 to about 1,000, such as from about 1 to about 100, such as from about 1 to about 20; and
z is a positive number such as about 1 or more, such as from about 1 to about 50, such as from about 1 to about 10, such as from about 1 to about 3.

The carbon chain CₓH_{y} can be a substituted hydrocarbyl or unsubstituted hydrocarbyl, substituted alkoxy or unsubstituted alkoxy, substituted aryl or unsubstituted aryl, polymeric or non-polymeric, saturated or unsaturated, linear or branched, cyclic or acyclic, aromatic or non-aromatic as described herein.

Illustrative, but non-limiting, examples of precursors of the organic group include, or be selected from, mercaptosuccinic acid (MSA), 3-mercaptopropionic acid (MPA), mercaptopoly(ethylene glycol) carboxylic acid, thioglycolic acid, thiolactic acid, mercaptoisobutyric acid, mercaptobutyric acid, mercaptohexanoic acid, cysteine, mercaptooctanoic acid, mercaptoundecanoic acid, mercaptododecanoic acid, mercaptohexadecanoic acid, homocysteine, N-acetyl-cysteine, glutathione, mercaptobenzoic acid, mercaptophenylacetic acid, or combinations thereof in any suitable proportion. The structures of MSA (1), MPA (2), and mercaptopoly(ethylene glycol) carboxylic acid (3) are

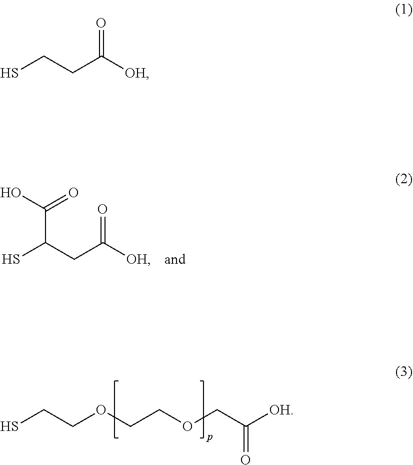

With respect to mercaptopoly(ethylene glycol) carboxylic acid (3), p is a positive number greater than about 1, such as about 2 or more, such as about 5 or more, such as from about 10 to about 1,000, such as from about 15 to about 500, such as from about 20 to about 200, such as from about 50 to about 150, such as from about 75 to about 125. In some aspects, the average number molecular weight (Mₙ) of the mercaptopoly(ethylene glycol) carboxylic acid can be from about 250 g/mol to about 100,000 g/mol, such as from about 450 g/mol to about 50,000 g/mol, such as from about 650 g/mol to about 25,000 g/mol, such as from about 900 g/mol to about 10,000, such as from about 2,000 g/mol to about 7,500, such as from about 3,000 g/mol to about 6,000 g/mol, such as from about 4,000 to about 5,000 g/mol.

The organic group can include, or be selected from, 3-mercaptopropionic acid, mercaptosuccinic acid, mercaptopoly(ethylene glycol) carboxylic acid, or combinations thereof.

In some aspects, the functionalized metal is represented by:

Mₘ(S—R—X¹)ₘ,

wherein m is a positive number from about 3×10² to about 1×10²¹, such as from about 3×10⁵ to about 1×10¹⁸, such as from about 3×10⁸ to about 1×10¹⁵, such as from about 3×10¹⁰ to about 1×10¹²;
n is a positive number from about 1 to about 1×10¹⁴, such as from about 1×10² to about 1×10¹², such as from about 1×10⁴ to about 1×10¹⁰, such as from about 1×10⁶ to about 1×10⁸;
and M, R, and X¹ are described above. The metal (M) is bound, chemically and/or physically (e.g., covalent, dative, ionic, etc.), to the sulfur atom of the organic group such that a metal-sulfur (M—S) bond is formed.

A ratio of m to n can be from about 300:1 to about 1×10⁷:1, such as from about 3×10³:1 to about 1×10⁶:1, such as from about 3×10⁵:1 to about 1×10⁵:1.

In at least one aspect, the organic group comprises, or is selected from,

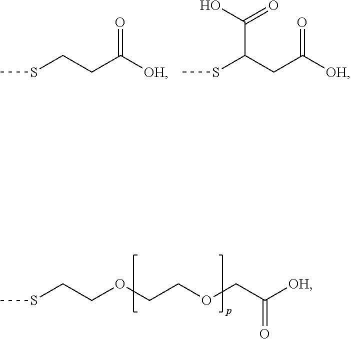

or combinations thereof, wherein:
“ [symbol] ” represents a bond to the Group 10 to Group 14 metal; and
p is a positive number greater than about 1, such as about 2 or more, such as about 5 or more, such as from about 10 to about 1,000, such as from about 15 to about 500, such as from about 20 to about 200, such as from about 50 to about 150, such as from about 75 to about 125.

The average number molecular weight (Mₙ) of the mercaptopoly(ethylene glycol) carboxylic acid can be those described above.

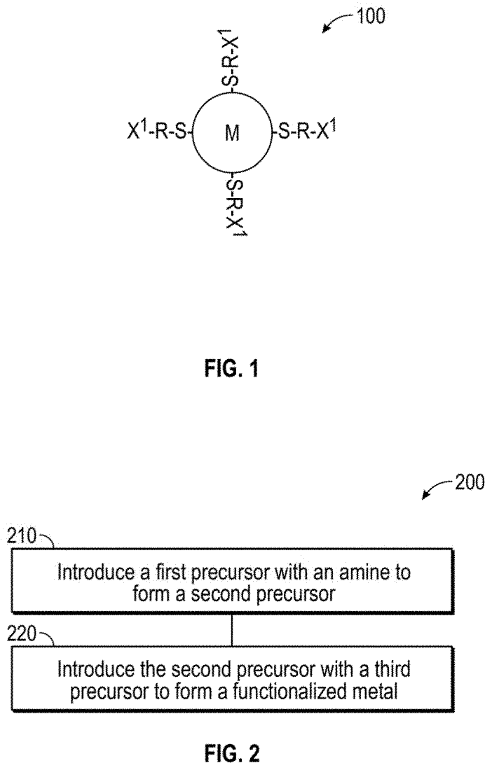

FIG. 1 shows an example illustration of a functionalized metal 100. Here, the organic group is a ligand of the central metal atom and is bound to metal by the sulfur atom. X¹ (e.g., the carboxyl group) is at the other end of the organic group and can be used to bind an analyte such as NH₃ for, e.g., sensing applications.

In some aspects, and when the functionalized metal is in the form of particles, an average particle size of the particles is from about 2 nm to about 2000 μm, such as from about 20 nm to about 200 μm, such as from about 200 nm to about 20 μm, such as from about 500 nm to about 2 μm. In at least one aspect, the average particle size of the particles is from about 1 nm to about 100 nm, such as from about 5 nm to about 50 nm, such as from about 10 nm to about 25 nm. The average particle size is measured by transmission electron spectroscopy (TEM). For spherical particles, the average particle size is the average diameter as measured by TEM, and for non-spherical particles, the average particle size is an equivalent edge length. The equivalent edge length is measured from TEM.

In some aspects, and when the functionalized metal is in the form of particles, an average particle surface area of the particles can be from about 12.56 nm² to about 1.256×10⁷ μm², such as from about 1256 nm to about 1.256×10⁵ μm, such as from about 12.56 μm to about 1.256×10³ μm. The average particle surface area of the particles is determined by the equation

surface area=4πr²

where r is the radius of the spherical particle as determined from the diameter according to TEM.

In some aspects, the functionalized metal can have a molar ratio of Group 10 to Group 14 metal(s) to sulfur atom(s) that is about 1:1 to about 1×10⁷:1, such as from about 1:1 to about 1×10⁵:1, such as from about 1:1 to about 1×10³:1 to about. In at least one aspect, the functionalized metal has a molar ratio of Group 10 to Group 14 metal(s) to sulfur atom(s) from about 10:1 to about 100:1, such as from about 20:1 to about 80:1, such as from about 30:1 to about 70:1, such as from about 40:1 to about 60:1. In other aspects, the molar ratio of Group 10 to Group 14 metal(s) to sulfur atom(s) is from about 1:1 to about 1000:1, such as from about 1:1 to about 800:1, such as from about 1:1 to about 500:1, such as from about 1:1 to about 200:1, such as from about 1:1 to about 50:1, such as from about 1:1 to about 5:1.

For the functionalized metal, the molar ratio of Group 10 to Group 14 to sulfur atom(s) is measured by transmission electron microscopy of the functionalized metal being analyzed.

For processes for producing a functionalized metal (described below), the molar ratio of the Group 10 to Group 14 metal(s) to sulfur atom(s) of the functionalized metal is determined based on the starting material molar ratio used for the synthesis.

### Processes for Producing Functionalized Metals

The present disclosure also relates to processes for forming functionalized metals such as those described above. FIG. 2 is a flowchart showing selected operations of an example process 200 for producing a functionalized metal according to at least one aspect of the present disclosure.

The process 200 can include introducing, under first conditions, a first precursor comprising one or more elements (e.g., metals) from Group 10 to Group 14 of the periodic table of elements with an amine to form a second precursor comprising the Group 10 to Group 14 metal at operation 210. The second precursor can be, e.g., an amine-stabilized metal. “Second precursor” and “amine-stabilized metal” are used interchangeably. The second precursor can be in the form of, e.g., nanoparticles.

As a non-limiting example, the second precursor can be an alkylamine-stabilized metal particle such as an alkylamine-stabilized gold particle. The amine can act as a solvent and/or a stabilizer.

According to at least some aspects, the first precursor comprising one or more elements from Group 10 to Group 14 of the periodic table of elements includes Ni, Pd, Pt, Cu, Ag, Au, Cd, Hg, or combinations thereof. The first precursor can also include one or more ligands. Such ligands can include, or can be selected from, halide (e.g., I⁻, Br⁻, Cl⁻, or F⁻), acetylacetonate (O₂C₅H₇⁻), hydride (H⁻), SCN⁻, NO₂⁻, NO₃⁻, N₃⁻, OH⁻, oxalate (C₂O₄²), H₂O, acetate (CH₃COO⁻), O₂⁻, CN⁻, OCN⁻, OCN⁻, CNO⁻, NH₂⁻, NH²⁻, NC⁻, NCS⁻, N(CN)₂⁻, pyridine (py), ethylenediamine (en), 2,2′-bipyridine (bipy), PPh₃, or combinations thereof. In some aspects, the first precursor can include metal acetates, metal acetylacetonates, metal halides, and/or metal nitrates. As illustrative, but non-limiting, examples, the first precursor can include, or be selected from, chloroauric acid (HAuCl₄), copper acetylacetonate Cu(O₂C₅H₇)₂, silver nitrate (AgNO₃), or combinations thereof.

The amine can be a primary amine, a secondary amine, a tertiary amine, or combinations thereof. The amine can include an unsubstituted hydrocarbyl or a substituted hydrocarbyl (as described above) bonded to the nitrogen of the amine, where the unsubstituted hydrocarbyl or substituted hydrocarbyl can be saturated or unsaturated, linear or branched, cyclic or acyclic, aromatic or non-aromatic. Illustrative, but non-limiting, examples of amines include oleylamine (OLA), octadecylamine (ODA), hexadecylamine (HDA), dodecylamine (DDA), tetradecylamine (TDA), or combinations thereof.

In some examples, the first precursor is introduced to more than one amine, where each amine is in suitable proportions relative to the first precursor. As non-limiting examples, the first precursor can be introduced to OLA and TDA, or DDA and TDA, or HDA and TDA, or OLA and HDA. Other amine combinations are contemplated.

For operation 210, the molar ratio of materials can be adjusted as desired. In some examples, the molar ratio of first precursor to amine(s) is from about 1:1 to about 1×10⁷:1, such as from about 1:1 to about 1×10⁵:1, such as from about 1:1 to about 1×10³:1, such as from about 1:1 to about 100:1, such as from about 1:1 to about 50:1, such as from about 1:1 to about 20:1, such as from about 1:1 to about 5:1.

The first conditions in operation 210 can include a reaction temperature and a reaction time. The reaction temperature of operation 210 can be from about 100° C. to about 320° C., such as from about 110° C. to about 310° C., such as from about 120° C. to about 300° C., such as from about 130° C. to about 290° C., such as from about 140° C. to about 280° C., such as from about 150° C. to about 270° C., such as from about 160° C. to about 260° C., such as from about 170° C. to about 250° C., such as from about 180° C. to about 240° C., such as from about 190° C. to about 230° C., such as from about 200° C. to about 220° C. In some aspects, the reaction temperature of operation 210 is from about 150° C. to about 280° C. or from about 180° C. to about 250° C. Higher or lower temperatures can be used when appropriate. The reaction time of operation 210 can be at least about 1 minute (min), such as from about 5 min to about 6 hours (h), such as from about 10 min to about 5.5 h, such as from about 15 min to about 5 h, such as from about 30 min to about 4 h, such as from about 45 min to about 3 h, such as from about 1 h to about 2 h. The reaction time of operation 210 can be more or less depending on, e.g., the level of conversion desired. Any reasonable pressure can be used during operation 210.

The first conditions of operation 210 can include stirring, mixing, and/or agitation to, e.g., ensure homogeneity of the mixture. The first conditions can also include performing the reaction under a non-reactive gas, such as N₂ and/or Ar. The mixture of the first precursor and the amine can be degassed with these or other non-reactive gases prior to, during, and/or after, adjusting the temperature. After a suitable time, the reaction mixture comprising the reaction product (e.g., the second precursor) of operation 210 can be subjected to filtration, separation, cleaning, quenching, washing, purification, and/or other suitable processes to remove undesired components and isolate the second precursor from the other components of the reaction mixture. For example, the mixture comprising the reaction product of operation 210 can be centrifuged to separate the second precursor particles from the mixture. Additionally, or alternatively, the second precursor can be washed with polar solvent(s), such as water, acetone, ethanol, methanol, or combinations thereof, and/or non-polar solvent(s), such as hexane, pentane, toluene, or combinations thereof. Other solvents for washing can include ether solvents such as diethyl ether and tetrahydrofuran; chlorocarbon solvents such as dichloromethane and chloroform; as well as ethyl acetate, dimethylformamide, acetonitrile, benzene, isopropanol, n-butanol, n-propanol. Mixtures of two or more solvents such as those listed above, in suitable proportions, can be utilized for washing or otherwise separating the second precursor from other components in the reaction mixture. As an example, a solvent can be added to the second precursor and centrifuged. The supernatant can be discarded and the remaining pellet can be dispersed in a suitable solvent or mixture of solvents such as those listed above. The resultant pellet and solvent(s) can then be centrifuged to obtain the second precursor.

The process 200 can further include introducing, under second conditions, the second precursor with a third precursor to form the functionalized metal at operation 220. Operation 220 can be referred to as a ligand exchange reaction. The functionalized metal can be a reaction product of operation 220 where the sulfur atom of the third precursor is bound to a metal. The third precursor includes, or is, the starting material that contains the organic group as described above. Illustrative, but non-limiting, examples of the third precursor include MSA, MPA, mercaptopoly(ethylene glycol) carboxylic acid, thioglycolic acid, thiolactic acid, mercaptoi sobutyric acid, mercaptobutyric acid, mercaptohexanoic acid, cysteine, mercaptooctanoic acid, mercaptoundecanoic acid, mercaptododecanoic acid, mercaptohexadecanoic acid, homocysteine, N-acetyl-cysteine, glutathione, mercaptobenzoic acid, mercaptophenylacetic acid, or combinations thereof in any suitable proportion. Other compounds comprising organic groups are described above.

For operation 220, the molar ratio of materials can be adjusted as desired. In some examples, the molar ratio of second precursor to third precursor is from about 1:1 to about 1×10⁷:1, such as from about 10:1 to about 1×10⁶:1, such as from about 100:1 to about 1×10⁵:1, such as from about 1×10³:1 to about 1×10⁴:1. In other aspects, the molar ratio of second precursor to third precursor is from about 1:1 to about 100:1, from about 1:1 to about 80:1, from about 1:1 to about 50:1, from about 1:1 to about 20:1, from about 1:1 to about 10:1, or from about 1:1 to about 5:1.

Operation 220 can include introducing the second precursor and the third precursor with a solvent such as those described above, such as water. The reaction mixture formed, which includes the second precursor, the third precursor, and an optional solvent can be stirred, mixed, and/or agitated to, e.g, ensure homogeneity of the mixture. The second conditions in operation 220 can include a reaction temperature and a reaction time. The reaction temperature of operation 220 can be from about 20° C. to about 320° C., such as from about 110° C. to about 310° C., such as from about 120° C. to about 300° C., such as from about 130° C. to about 290° C., such as from about 140° C. to about 280° C., such as from about 150° C. to about 270° C., such as from about 160° C. to about 260° C., such as from about 170° C. to about 250° C., such as from about 180° C. to about 240° C., such as from about 190° C. to about 230° C., such as from about 200° C. to about 220° C. Higher or lower temperatures can be used when appropriate. The reaction time for operation 220 can be at least about 1 minute (min), such as from about 5 min to about 48 h, such as from about 10 min to about 24 h, such as from about 30 min to about 10 h, such as from about 1 h to about 8 h, such as from about 2 h to about 7 h, such as from about 3 h to about 6 h, such as from about 4 h to about 5 h. The reaction time for operation 220 can be more or less depending on, e.g., the level of conversion desired. Any reasonable pressure can be used during the reaction to form the functionalized metal of operation 220.

The second conditions can also include performing the reaction under a non-reactive gas, such as N₂ and/or Ar. The mixture of the second precursor, the third precursor, and the optional solvent, can be degassed with these or other non-reactive gases prior to, during, and/or after, adjusting the temperature. After a suitable time, the reaction mixture comprising the reaction product of operation 220 (e.g., the functionalized metal) can be subjected to filtration, separation, cleaning, quenching, washing, purification, and/or other suitable processes to remove undesired components and isolate the functionalized metal from the other components of the reaction mixture. For example, the mixture comprising the reaction product of operation 220 can be centrifuged to separate the functionalized metal (which may be in the form of particles) from the mixture. Additionally, or alternatively, the functionalized metal can be washed with polar solvent(s), such as water, acetone, ethanol, methanol, or combinations thereof, and/or non-polar solvent(s), such as hexane, pentane, toluene, or combinations thereof. Other solvents for washing can include ether solvents such as diethyl ether and tetrahydrofuran; chlorocarbon solvents such as dichloromethane and chloroform; as well as ethyl acetate, dimethylformamide, acetonitrile, benzene, isopropanol, n-butanol, n-propanol. Mixtures of two or more solvents such as those listed above, in suitable proportions, can be utilized for washing or otherwise separating the functionalized metal from other components in the reaction mixture. As an example, a solvent can be added to the functionalized metal and centrifuged. The supernatant can be discarded and the remaining pellet can be dispersed in a suitable solvent or mixture of solvents such as those listed above. The resultant pellet and solvent(s) can then be centrifuged to obtain the functionalized metal.

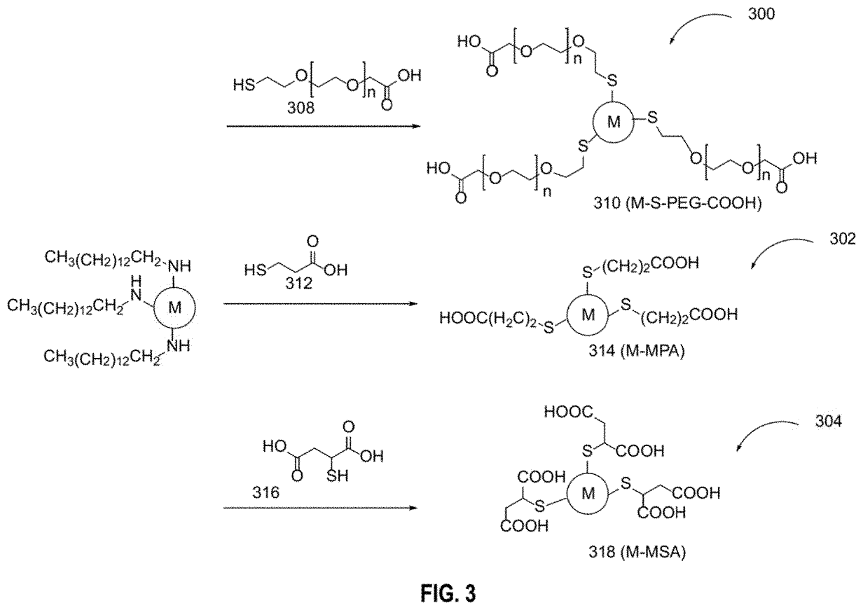

FIG. 3 shows various reaction diagrams for the conversion of operation 220, e.g., the conversion of the second precursor 306 to functionalized metals. The reaction diagrams, starting materials, reactants, and products shown in FIG. 3 are illustrative, but non-limiting, examples. Reaction diagram 300 illustrates the reaction of the second precursor 306 with mercaptopoly(ethylene glycol) carboxylic acid (SH-PEG-COOH) 308 to form functionalized metal 310 (M-S-PEG-COOH). As another example, reaction diagram 302 illustrates that the amine of the second precursor 306 can undergo ligand exchange with 3-mercaptopropionic acid (MPA) 312 to produce functionalized metal 314 (M-MPA). As another example, reaction diagram 304 shows a ligand exchange between the amine of the second precursor 306 with mercaptosuccinic acid (MSA) 316 to produce functionalized metal 314 (M-MSA).

### Uses of Functionalized Metals

The present disclosure also relates to uses of the functionalized metals described herein. In some aspects, the functionalized metal can be used in gas sensing applications and/or can be incorporated into devices (e.g., gas sensor devices, chemoresistive sensors) useful for such applications. In some aspects, the functionalized metal can be utilized to detect, monitor, measure, determine, or otherwise sense, the presence of analytes (such as amines, such as NH₃) in a variety of settings or environments. As an example, the functionalized metal can be incorporated into a chemoresistive sensor to detect, monitor, measure, determine, or otherwise sense, the presence of NH₃ formed in, e.g., the catalytic converter of an automobile. In addition to NH₃, engine exhaust contains various gases such as CO, CO₂, O₂, N₂O, NO, NO₂, H₂O, and CH₄. Cross interference effects from these gases can be detrimental to NH₃ sensing. As described herein, use of the functionalized metal can overcome such cross-interference effects due to, e.g., its selectivity for NH₃. Such sensors can also be used in other transportation applications such as other land vehicles (trucks), trains, aircraft, and watercraft.

As another example, the functionalized metal can be incorporated into field-portable monitors for environmental monitoring. Here, the sensitivity and selectivity of the functionalized metal enables detection, monitoring, measurement, determining, or otherwise sensing, of trace amounts of an analyte. As another example, the functionalized metal can be incorporated into chemoresistive sensors useful in personal protective equipment, industrial settings, and other applications, as an alert for the presence of an analyte. In these and other applications, the functionalized metal can be the sensing material of a sensor or the sensing material of the sensor can include the functionalized metal.

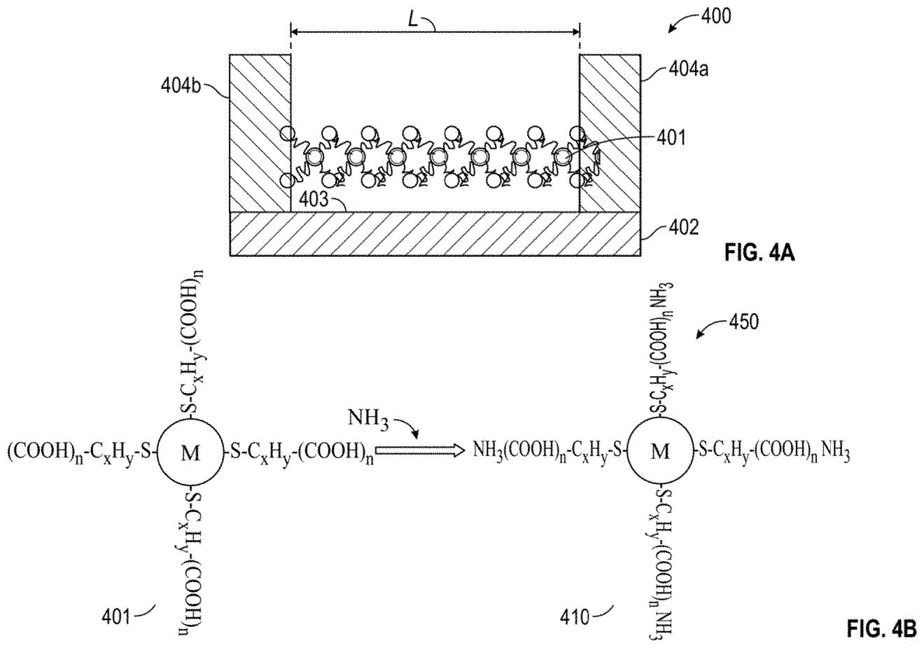

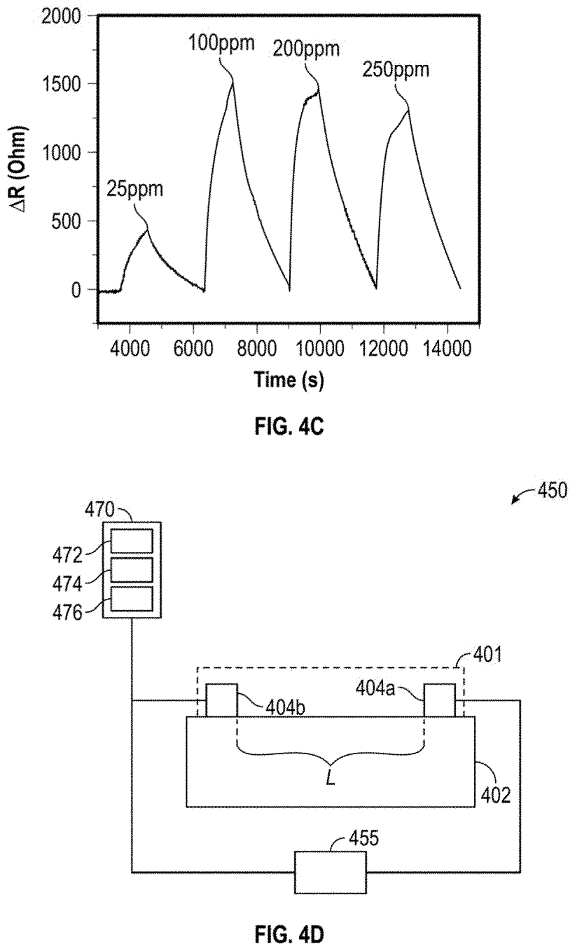

FIG. 4A shows an illustration of a side view of an example device 400 (e.g., a chemoresistive sensor) for detecting, monitoring, measuring, determining, or otherwise sensing, the presence of an analyte. The device 400 includes a substrate 402. Illustrative, but non-limiting examples of substrates 402 include mica, quartz, silicon, SiO₂, any suitable plastic such as polycarbonate, polystyrene, or combinations thereof. The substrates can be doped with nitrogen, boron, and/or aluminum. Electrodes 404a and 404b are disposed on at least a portion of the surface 403 of the substrate 402. Electrode 404a can be the source electrode and electrode 404b can be the drain electrode, or vice-versa. The electrodes 404a and 404b are separated by a distance, L, where L is from about 10 nm to about 1 mm, such as from about 100 nm to about 100 μm, such as from about 500 nm to about 5 μm. The electrodes 404a and 404b can be made of, or include, any suitable conductive material such as graphene, glassy carbon, copper, gold, chromium, nickel, silver, titanium, or combinations thereof. The electrodes 404a and 404b can be fabricated using electron beam lithography according to known methods. A functionalized metal 401 can be disposed between the electrodes 404a and 404b and disposed on a surface 403 of the substrate 402. Additionally, or alternatively, the functionalized metal 401 can be disposed on at least a portion of the electrodes. The functionalized metal 401 can be in the form of particles (nanoparticles, microparticles, or macroparticles), a monolayer film, a multilayer film (having from about 2 to about 10,000 layers, such as from about 20 layers to about 1000 layers, such as from about 100 layers to about 500 layers), or other suitable structure.

The functionalized metal 401 can be deposited using various techniques. Deposition techniques can include syringe coating, dip coating, knife edge coating, and/or spin coating, micro-inject coating, and 3D print coating to produce a particle, a film, or other suitable structure on the substrate 402 and/or electrodes. For deposition, the functionalized metal 401 can be in the form of a solution or suspension in a solvent such as a hydrophilic solvent, such as water, ethanol, and/or acetone. After depositing the functionalized metal 401, the device 400 can be dried by air drying at room temperature (e.g., about 15° C. to about 25° C.) or heated in an oven to evaporate the solvent.

The functionalized metal 401 acts as a sensing material that changes its electrical resistance in response to variations in the chemical environment around the device 400. The change in the resistance of the functionalized metal 401, between the absorbed state and desorbed state, can be used to detect, monitor, measure, determine, or otherwise sense, an analyte such as NH₃.

In operation, a voltage is applied to the electrodes 404a, 404b of the device 400 and the current is measured. Based on the applied voltage and the measured current, the resistance change of the functionalized metal 401 can be calculated using Ohm's law. FIG. 4B shows a pictorial representation of NH₃ sensing by the functionalized metal 401. When the functionalized metal 401 is exposed to an atmosphere containing NH₃, the NH₃ interacts (via, e.g., bonding or absorption) with the carboxylic acid portions of the functionalized metal 401 as shown in chemical structure 410. The bonding or absorption of NH₃ to the functionalized metal 401 causes the resistance of the functionalized metal 401 to change. FIG. 4C shows exemplary data for the resistance change (ΔR) of the functionalized metal 401. For the example shown in FIG. 4C, the sensor device was evaluated by determining ΔR of example MSA—modified Au nanoparticles in the presence of various concentrations of NH₃ pulsed at selected time points.

FIG. 4D shows an example device 450 (e.g., chemiresistive sensor) according to another aspect of the present disclosure. The device 450 can be used to detect, monitor, measure, determine, or otherwise sense, the presence of an analyte. The device 450 includes a substrate 402 having electrodes 404a, 404 disposed thereon. The substrate 402 and electrodes 404a, 404b can be made of, or include, those materials described above. The electrodes 404a, 404b are separated by a distance, L, where L is from about 10 nm to about 1 mm, such as from about 100 nm to about 10 μm, such as from about 500 nm to about 5 μm. Larger or smaller distances, L, are contemplated. The functionalized metal 401 can be disposed on at least a portion of the substrate 402 and/or on at least a portion of the electrodes 404a, 404b. Although the functionalized metal 401 is shown to surround both electrodes 404a, 404b, the functionalized metal 401 can be disposed between the electrodes 404a, 404b and/or be disposed on at least a portion of the electrodes 404a, 404b. The functionalized metal 401 can be in the form of particles (nanoparticles, microparticles, or macroparticles), a monolayer film, a multilayer film (having from having from about 2 to about 10,000 layers, such as from about 20 layers to about 1,000 layers, such as from about 100 layers to about 500 layers), or other structure. The functionalized metal 401 can be deposited by suitable methods such as those techniques described above.

In operation, a voltage is applied to the electrodes 404a, 404b of the device 450 and the current is measured. Based on the applied voltage and the measured current, the resistance of the functionalized metal 401 in the presence of an analyte can be calculated using Ohm's law.

As shown, the device 450 can further include a controller 470, the controller being electrically coupled to various elements of the device 450. The controller is used to measure the current flowing through the functionalized metal 401 and calculate a resistance (R) of the functionalized metal 401. The controller 470 can also be utilized to control the voltage source 455. The controller 470 can also be used to send a signal to an input/output device, such as a display unit or an audio device (not shown) indicating a resistance change. The controller 470 can include a processor 472, memory 474, and support circuits 476.

The processor 472 may be one of any suitable form of general purpose microprocessor, or a general purpose central processing unit (CPU), each of which can be used in an industrial setting, such as a programmable logic controller (PLC), supervisory control and data acquisition (SCADA) systems, or other suitable industrial controller. The memory 474 is non-transitory and may be one or more of readily available memory such as random access memory (RAM), read only memory (ROM), or any other form of digital storage, local or remote. The memory 474 contains instructions, that when executed by the processor 472, can facilitate one or more operations of, e.g., applying a voltage, the current measurement, the resistance calculation. The instructions in the memory 474 are in the form of a program product such as a program that implements the method of the present disclosure. The program code of the program product may conform to any one of a number of different programming languages. Illustrative computer-readable storage media include, but are not limited to: (i) non-writable storage media (e.g., read-only memory devices within a computer such as CD-ROM disks readable by a CD-ROM drive, flash memory, ROM chips, or any type of solid-state non-volatile semiconductor memory) on which information is permanently stored; and (ii) writable storage media (e.g., floppy disks within a diskette drive or hard-disk drive or any type of solid-state random-access semiconductor memory) on which alterable information is stored. Such computer-readable storage media, when carrying computer-readable instructions that direct the functions of the methods described herein, are examples of the present disclosure. In one example, the disclosure may be implemented as the program product stored on a computer-readable storage media (e.g., memory 474) for use with a computer system (not shown). The program(s) of the program product define functions of the disclosure, described herein.

The following examples are put forth so as to provide those of ordinary skill in the art with a complete disclosure and description of how to make and use aspects of the present disclosure, and are not intended to limit the scope of aspects of the present disclosure. Further, while the present disclosure refers to “nanoparticles”, it will be appreciated that the disclosure may be applied to particles having a larger size (e.g., “microparticles” and “macroparticles”). Similarly, while the present disclosure refers to films, it will be appreciated that the disclosure may be applied to layer(s), monolayer films, and multilayer films. In addition, efforts have been made to ensure accuracy with respect to numbers used (e.g. amounts, dimensions, etc.) but some experimental errors and deviations should be accounted for.

### EXAMPLES

Chloroauric acid (HAuCl₄), copper acetylacetonate, silver nitrate (AgNO₃), OLA, DDA, HDA, ODA, mercaptopoly(ethylene glycol) carboxylic acid (SH-PEG-COOH), 3-mercaptopropionic acid (MPA), and mercaptosuccinic acid (MSA) were purchased from Sigma Aldrich. All chemicals were used as received.

X-ray diffraction patterns were obtained using a Bruker D8 Advance X-ray diffractometer with Cu Ka radiation operated at a tube voltage of 40 kV and a current of 40 mA. Surface morphologies were investigated using a QUANTA™ FEG 650 scanning electron microscope (from FEI Tecnai) with a field emitter as the electron source. Transmission electron microscopy (TEM) images were captured using an FEI Tecnai 20 microscope with an accelerating voltage of 200 kV. FT-IR spectra were obtained using a NEXUS 670 ThermoNicolet FT-IR. For characterization by FT-IR, sample suspensions were drop casted onto KBr crystal plates and allowed to dry as a film prior to the measurement.

A UV-Vis spectrometer (Cary 5000) was used to record the extinction spectra of the metal nanoparticles. For characterization by UV-Vis spectrometry, solvents sufficient to disperse the functionalized metal (e.g., functionalized metal nanoparticles) were used such as hydrophobic solvents, such as hexane, toluene, and/or chloroform.

### Example 1

### Synthesis of the Second Precursor

Example 1A. Gold Nanoparticles: HAuCl₄ (50 mg) was mixed with oleylamine (3 mL) and tetradecylamine (3 g) in a flask under an Ar and/or N₂ environment to form a solution/suspension. After degassing for 10 minutes, the solution/suspension was heated to 150° C. under Ar and/or N₂. After keeping the solution/suspension at this temperature for 60 minutes, the solution was cooled to room temperature. The products were isolated by centrifugation at 6000 rpm for 15 minutes, and the supernatant was discarded. The pellet was then washed (2×) with hexane and ethanol, sequentially. The second precursor (e.g., gold nanoparticles) was stored in a hydrophobic solvent (e.g., hexane, toluene, and/or chloroform). The gold nanoparticles had a molar ratio of gold to amine of about 4:1.

Example 1B. Silver Nanoparticles: AgNO₃ (51 mg) was mixed with oleylamine (3 mL) and tetradecylamine (3 g) in a flask under an Ar and/or N₂ environment to form a solution/suspension. After degassing for 10 minutes, the solution/suspension was heated to 150° C. under Ar and/or N₂. After keeping the solution/suspension at this temperature for 60 minutes, the solution was cooled to room temperature. The products were isolated by centrifugation at 6000 rpm for 15 minutes, and the supernatant was discarded. The pellet was then washed (2×) with hexane and ethanol, sequentially. The second precursor (e.g., silver nanoparticles) were stored in a hydrophobic solvent (e.g., hexane, toluene, and/or chloroform). The silver nanoparticles had a molar ratio of silver to amine of about 4:1.

Example 1C. Copper Nanoparticles: Copper acetylacetonate (52 mg) was mixed with oleylamine (3 mL) and tetradecylamine (3 g) in a flask under an Ar and/or N2 environment to form a solution/suspension. After degassing for 10 minutes, the solution/suspension was heated to 150° C. under Ar and/or Na. After keeping the solution/suspension at this temperature for 60 minutes, the solution was cooled to room temperature. The products were isolated by centrifugation at 6000 rpm for 15 minutes, and the supernatant was discarded. The pellet was then washed (2×) with hexane and ethanol, sequentially. The second precursor (e.g., copper nanoparticles) were stored in a hydrophobic solvent (e.g., hexane, toluene, and/or chloroform). The copper nanoparticles had a molar ratio of copper to amine of about 4:1.

### Example 2

### Synthesis of Functionalized Metals

The second precursor, e.g., gold nanoparticles (10 mg) from Example 1A, were mixed with mercaptosuccinic (2 mL) acid and water (5 mL), and stirred for 8 hours under an Ar and/or N₂ environment while monitoring ligand exchange. The resulting products were washed twice with deionized water by centrifugation at 4000 rpm for 5 minutes. The functionalized metal (MSA-modified Au nanoparticles) were stored in a hydrophilic solvent (e.g., water, acetone, ethanol, and/or methanol) before characterization. Similar procedures were utilized to convert the silver-amine nanoparticles (Example 1B) and copper-amine nanoparticles (Example 1C) to their respective functionalized metal nanoparticles.

Metal nanoparticles modified with different functional groups were also synthesized. Here, similar procedures were used to convert the second precursor to 3-mercaptopropioninc acid-modified Au nanoparticles (MPA-modified Au nanoparticles) and to mercaptopoly(ethylene glycol) carboxylic acid-modified Au nanoparticles (SH-PEG-COOH modified Au nanoparticles).

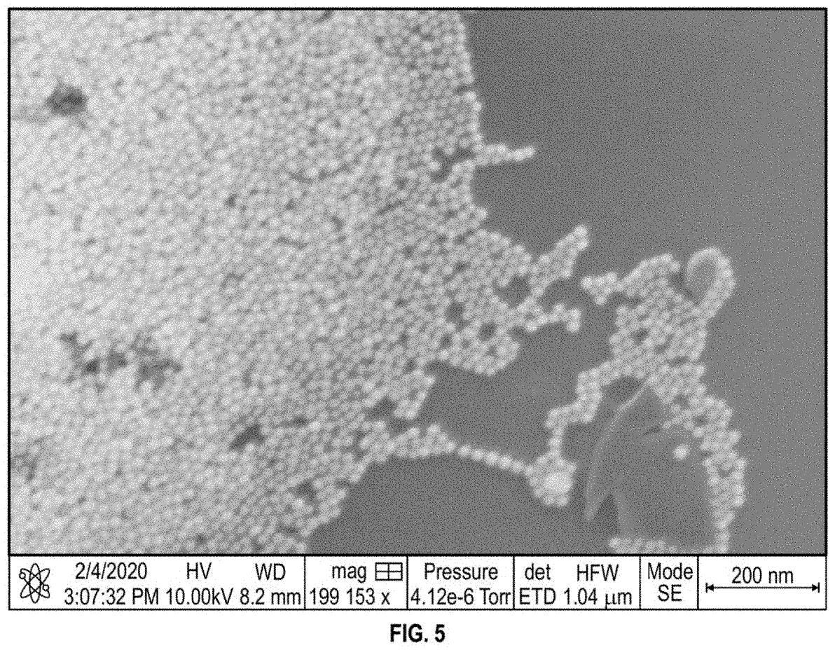

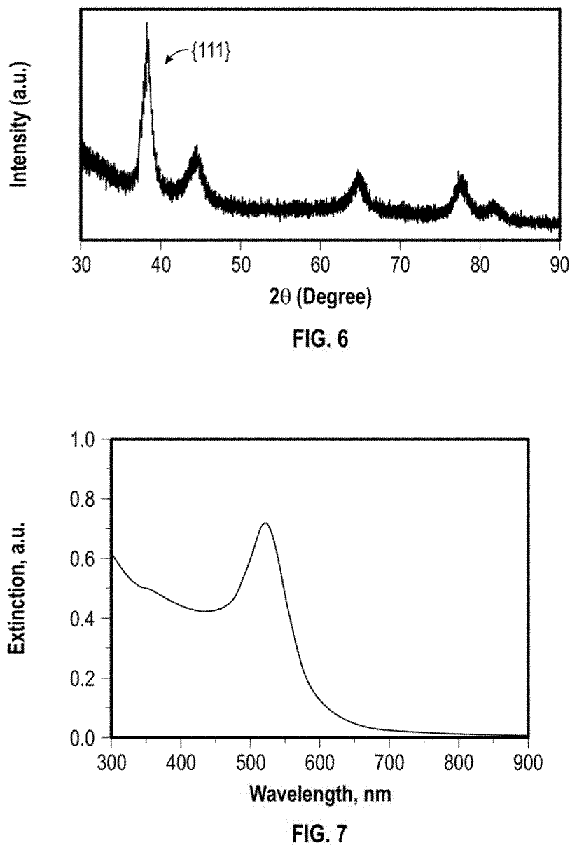

FIG. 5 is a TEM image of example MSA-modified Au nanoparticles. The TEM image indicates that the majority of products are spherical nanoparticles with an average particle diameter of about 12 nm. No obvious morphology and size change was found after replacing the alkylamines with ligands (e.g., MSA). FIG. 6 shows an XRD pattern of the example MSA-modified Au nanoparticles. The example MSA-modified Au nanoparticles have a strong and broad {111} diffraction peak. The XRD results indicate that the example MSA-modified Au nanoparticles are stable in air as there was no obvious phase change or oxidation after ligand exchange.

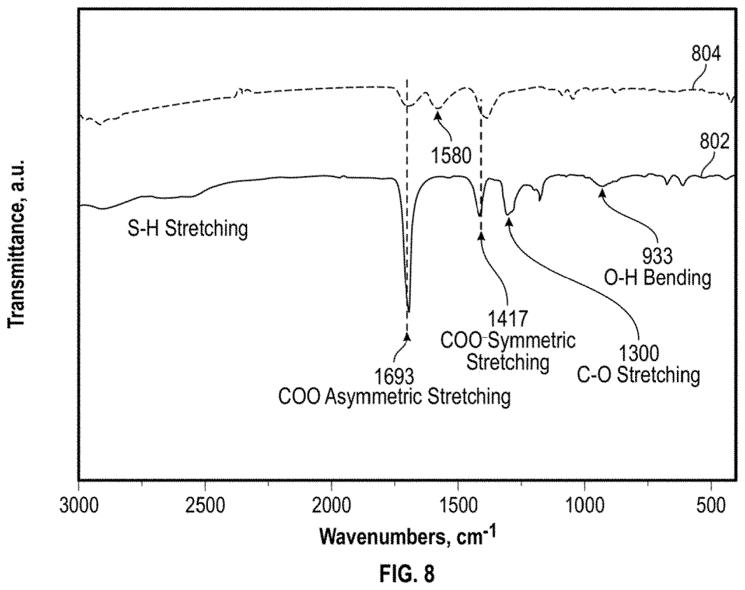

FIG. 7 is a UV-Vis absorption spectrum of the example MSA-modified Au nanoparticles dispersed in ethanol. The absorption peak of the example MSA-modified Au nanoparticles is centered at about 525 nm, which is consistent with their size. FT-IR was utilized to characterize the surface chemistry of the as-synthesized example MSA-modified Au nanoparticles, as shown in FIG. 8. FT-IR spectra show obvious differences between MSA 802 and the example MSA-modified Au nanoparticles 804. The FT-IR spectrum of MSA 802 shows characteristic COO⁻ symmetric and asymmetric stretching modes (bands at about 1693 cm⁻¹ and about 1417 cm⁻¹), C—O stretching mode (band at about 1300 cm⁻¹), O—H bending mode (band at about 933 cm⁻¹), and S—H stretching mode (band between about 2500 cm⁻¹ and about 2600 cm⁻¹). After ligand exchange, the FT-IR spectrum of the example MSA-modified Au nanoparticles 804 shows that the S—H band disappeared, while the characteristic COO⁻ bands remained. These results indicate that the sulfur atom is bound to the metal and that the example MSA-modified Au nanoparticles 804 have carboxyl groups.

### Example 3

### Preparation of Sensor Device

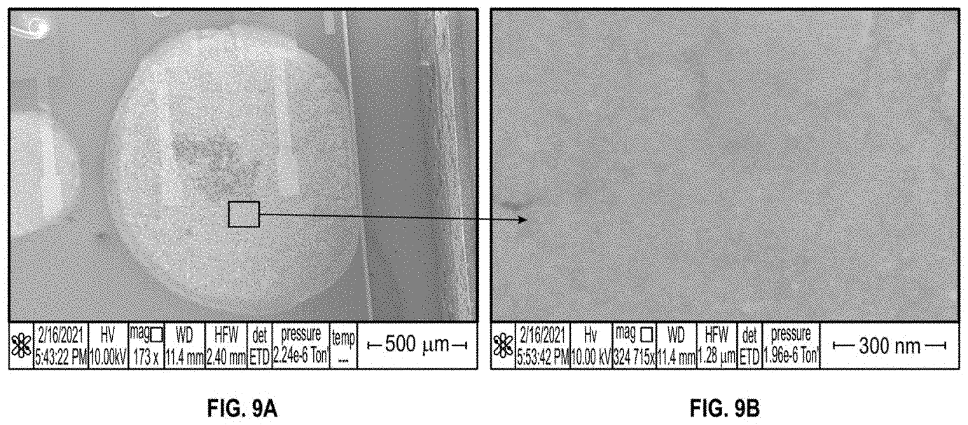

A sensor device (e.g., device 400) was fabricated with Cr/Au source and drain electrodes 404a, 404b patterned using electron beam lithography on the substrate 402. The substrate 402 for this example was a degenerately doped silicon substrate and used as a backgate. A solution/suspension of the functionalized metal 401 (in this example, MSA-modified Au nanoparticles) is deposited on portions of the substrate 402 and/or electrodes 404a, 404b via micropipette and microsyringe in the form of a solution or suspension in water or other hydrophilic solvent, and then dried at about 15° C. to about 25° C. FIG. 9A and FIG. 9B indicate that the film formed from the example MSA-modified Au nanoparticles is uniform and consistent with monodispersed functionalized Au nanoparticles. Sensor devices incorporating other functionalized metals were also fabricated by similar procedures.

Resistance Measurements: The source-drain current (I) was measured as a function of bias voltage and gate voltage applied to the electrodes 404a, 404b under ambient laboratory conditions. Based on the bias voltage (V) (or applied voltage) of about 5 V and the source-drain current (I), resistance (R) or resistance change (ΔR) of the functionalized metal 401 can then be calculated using Ohm's law: R=V|I.

### Example 4

### Performance

All gas sensing measurements were performed within a stainless steel gas chamber in the background of N₂ at room temperature. The mixtures containing different concentrations of the target gas were prepared by diluting the target gas with N₂. The total flow rate was maintained at 200 cm³/min with an automated gas delivery system operated by mass flow controllers. The sensors were pretreated in N₂ for 2 h before each measurement to acquire a stable baseline. The resistances of the sensors were recorded by a Keysight 34980A multifunction switch/measurement unit. The response value was defined as the ratio between the resistance change of a sensor (ΔR) in the presence of the target gas and the baseline resistance in N₂ (R0). The response and recovery time were defined as the time required to reach 90% of its final stable reading upon exposure or removal of the target gas. The voltage applied was 5 V.

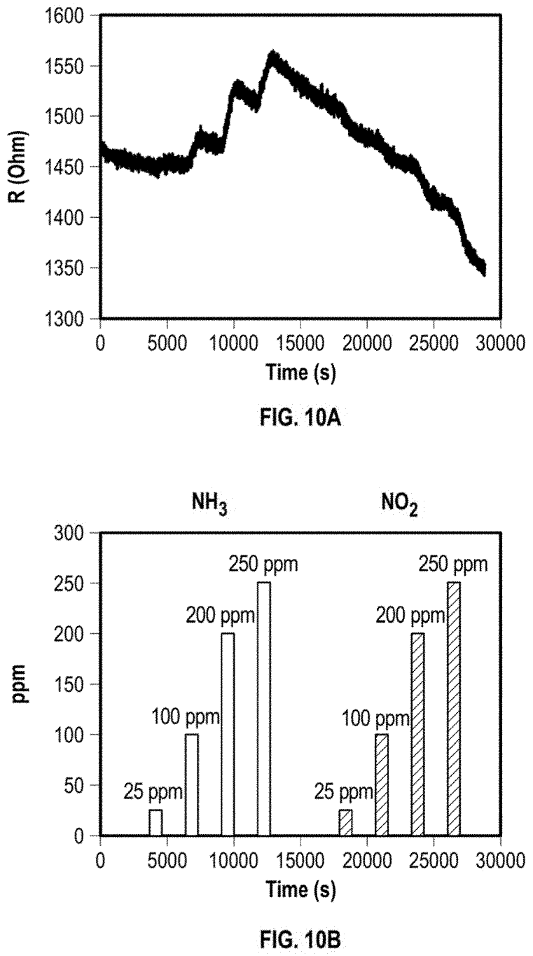

Sensitivity of the sensor was evaluated by investigating the between gas (NH₃ and NO₂) concentration and the magnitude of the sensor response. FIG. 10A shows resistance versus time data for an example sensor device in the presence of NH₃ and NO₂. The sensor device includes example MSA-modified Au nanoparticles as the sensing material. The resistance of the sensing material was measured at various concentrations of NH₃ gas and NO₂ gas pulsed at selected time points (FIG. 10B). As shown in FIG. 10A, the resistance for the example MSA-modified Au nanoparticles increases after exposures to ˜25 ppm, ˜100 ppm, ˜200 ppm, ˜250 ppm of NH₃ in N₂, while the resistance decreases after exposures to ˜25 ppm, ˜100 ppm, ˜200 ppm, ˜250 ppm of NO₂ in N₂. The results of FIG. 10A indicate that MSA-modified Au nanoparticles are useful for detecting NH₃.

While not wishing to be bound by theory, it is believed that the resistance increases because the NH₃ molecules coordinate with the carboxyl groups of the example MSA-modified Au nanoparticles. Such coordination increases the average distance between two nanoparticles which the electrons must tunnel through when a voltage is applied and electric current flows, thereby increasing the resistance. The increased resistance upon exposure to NH₃ and the decreased resistance upon exposure to NO₂ indicates the selectivity of the example MSA-modified Au nanoparticles toward NH₃.

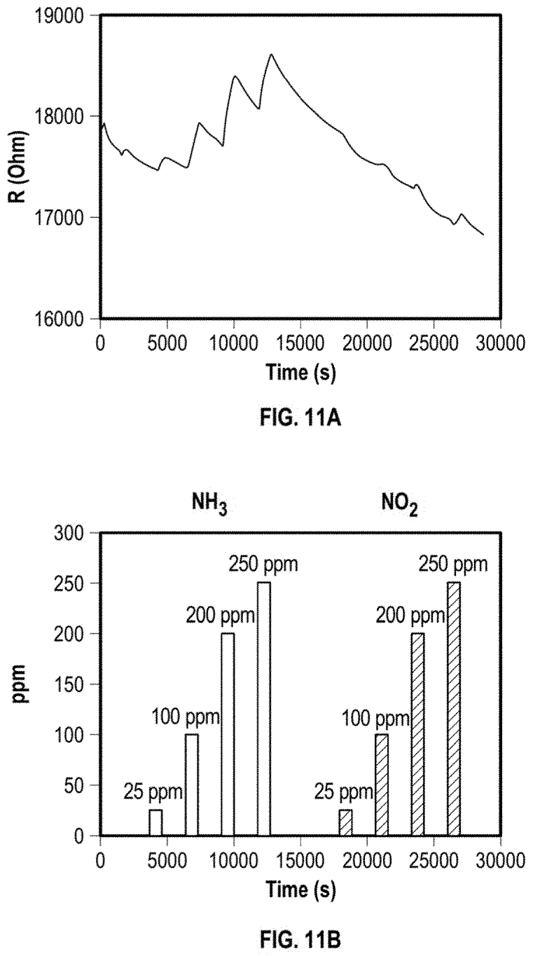

FIG. 11A shows resistance versus time data for an example sensor device in the presence of NH₃ and NO₂. The sensor device includes example 3-mercaptopropioninc acid-modified Au nanoparticles (MPA-modified Au nanoparticles) as the sensing material. The resistance of the sensing material was measured at various concentrations of NH₃ gas and NO₂ gas pulsed at selected time points (FIG. 11B). The results of FIG. 11A indicate that MPA-modified Au nanoparticles are useful for detecting NH₃.

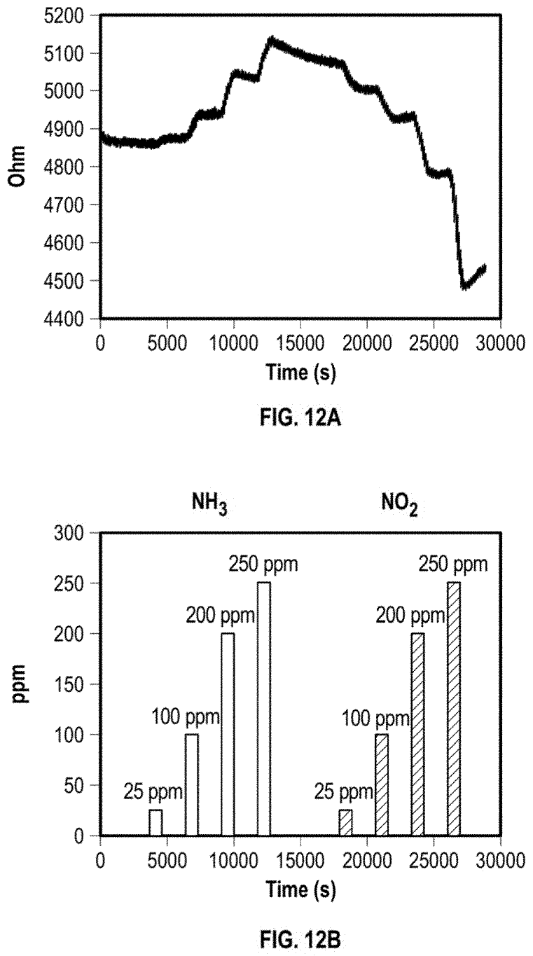

FIG. 12A shows resistance versus time data for an example sensor device in the presence of NH₃ and NO₂. The sensor device includes example mercaptopoly(ethylene glycol) carboxylic acid-modified Au nanoparticles (SH-PEG-COOH modified Au nanoparticles) as the sensing material. The resistance of the sensing material was measured at various concentrations of NH₃ gas and NO₂ gas pulsed at selected time points (FIG. 12B). The results of FIG. 12A indicate that SH-PEG-COOH modified Au nanoparticles is useful for detecting NH₃

In addition, FIGS. 10A, 11A, and 12A indicate that MSA-modified Au nanoparticles (FIG. 10A) are more sensitive to NH₃ than MPA-modified Au nanoparticles or SH-PEG-COOH modified Au nanoparticles. While not wishing to be bound by theory, it is believed that detection of NH₃ is through the interaction between NH₃ and the carboxyl group of the functionalized metal. Because each MSA molecule includes two carboxyl groups, more interactions between NH₃ and the carboxyl groups can be observed resulting in higher sensitivity to NH₃.

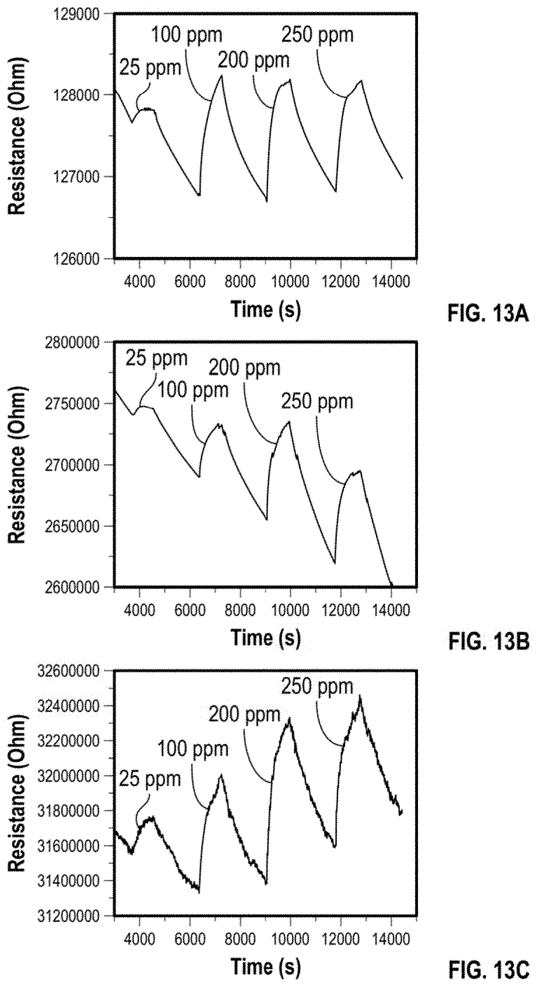

FIG. 13A-13C show exemplary resistance data of sensor devices incorporating example MSA-modified metal nanoparticles in the presence of NH₃ where the metal is varied.

Specifically, FIG. 13A shows resistance data where the sensing material includes MSA-modified Au nanoparticles, FIG. 13B shows resistance data where the sensing material includes MSA-modified Ag nanoparticles, and FIG. 13C shows resistance data where the sensing material includes MSA-modified Cu nanoparticles. For each sensor device, the resistance increased after exposures to ˜25, ˜100, ˜200, and ˜250 ppm of NH₃ in N₂. The results indicate that functionalized Au, Ag, and Cu nanoparticles are sensitive to NH₃ and that the functionalized metal of sensor devices can be fabricated from a variety of metals.

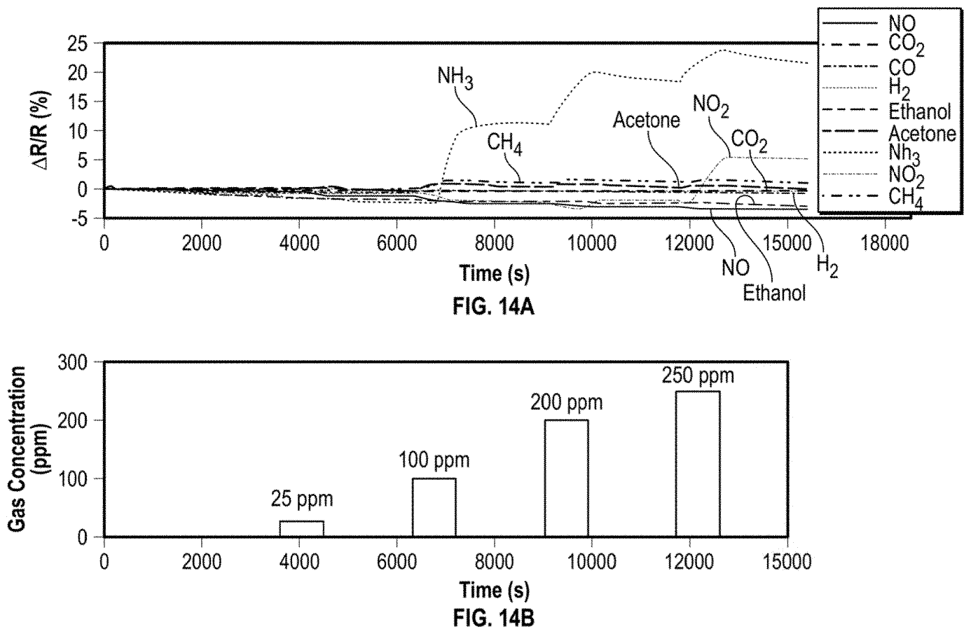

The selectivity of a sensor device incorporating MSA-modified nanoparticles was also evaluated by exposing the sensor device to various gases—NO, CO₂, CO, H₂, ethanol, acetone, NH₃, NO₂, and CH₄—at concentrations of ˜25 ppm, ˜100 ppm, ˜200 ppm, and ˜250 ppm in N₂. FIG. 14A shows the resistance change (normalized resistance, ΔR/R0 (%)) versus time of the sensor device after such exposures, and FIG. 14B shows the time points at which the gases were pulsed. The resistance data indicates that the functionalized metal nanoparticles described herein are selective for NH₃ over other gases.

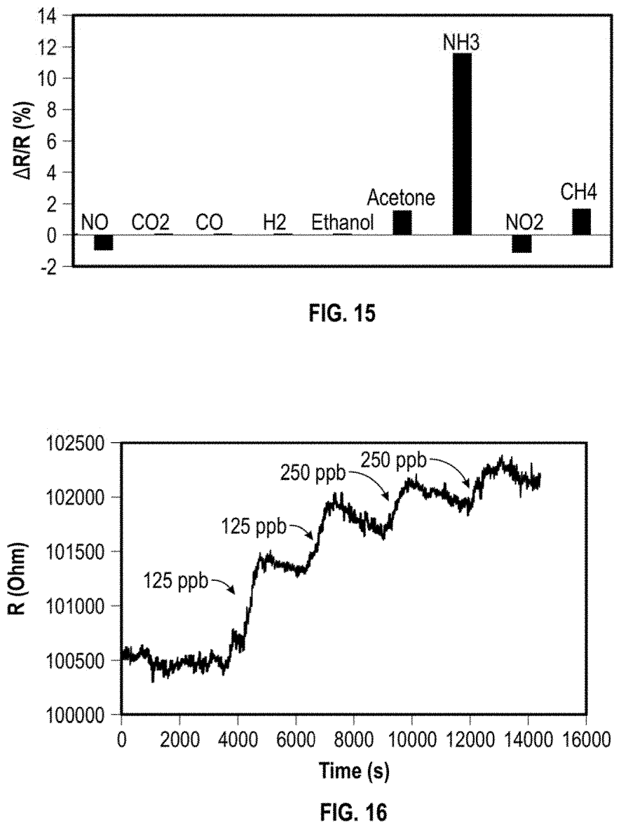

FIG. 15 shows the response to NH₃ versus the response to NO, CO₂, CO, H₂, ethanol, acetone, NH₃, NO₂, and CH₄. For this experiment, the concentration of each gas was about 100 ppm in N₂, and the sensor device includes MSA-modified Au nanoparticles as the sensing material. The results of FIG. 15 indicate that the MSA-modified Au nanoparticles is selective for NH₃ versus over the other analytes/gases tested.

FIG. 16 shows resistance data of a sensor device where the sensing material includes example MSA-modified Au nanoparticles. For this experiment, the sensor device was exposed to pulses of NH₃ (˜125 ppb, ˜125 ppb, ˜250 ppb, and ˜250 ppb) in N₂ at selected time points. The data shown in FIG. 16 indicates that the MSA-modified Au nanoparticles are very sensitive to NH₃, and detecting NH₃ concentrations of about 125 ppb or lower. As a comparative example, cobalt porphyrin-carbon nanotube composites, which are known in the art, have a detection limit of 500 ppb NH₃. The example functionalized metals described herein provide superior detection limits relative to such cobalt porphyrin-carbon nanotube composites. Moreover, nanostructures conventionally used as sensing materials typically exhibit long-term instability, whereas the functionalized metals described herein have long-term stability.

Functionalized metals, syntheses of functionalized metals, and uses of functionalized metals in, e.g., sensor devices are described herein. The functionalized metals exhibit high-performance detection of NH₃. Syntheses of the functionalized metals can be easily scaled-up, with lower production costs, relative to nanostructure-based NH₃ sensing materials. The functionalized metals are also fabricated by simpler methods than such nanostructures. Moreover the functionalized metals described herein, and sensor devices incorporating functionalized metals, can operate at room temperature with high sensitivity and high selectivity, whereas conventional materials incorporating semiconductive metal oxides must operate at temperatures above 400° C. for high NH₃ selectivity. Overall, the functionalized metals show, e.g., high selectivity, sub-ppm sensitivity, as well as high stability to air, moisture, and time.

### Aspects Listing

The present disclosure provides, among others, the following aspects, each of which can be considered as optionally including any alternate aspects:

Clause 1. A process for producing a functionalized metal, comprising:
introducing, under first conditions, a first precursor comprising a Group 10 to Group 14 metal with an amine to form a second precursor comprising the Group 10 to Group 14 metal; and
introducing, under second conditions, the second precursor with a third precursor to form the functionalized metal, the third precursor comprising an organic material having the formula:

HS—R—COOH,
wherein R is an unsubstituted hydrocarbyl, a substituted hydrocarbyl, an unsubstituted alkoxy, or a substituted alkoxy.

Clause 2. The process of Clause 1, wherein the functionalized metal has the formula:

Mₘ(S—R—COOH)ₙ,
wherein:
   M is the Group 10 to Group 14 metal; and
   a ratio of m to n is from about 300:1 to about 1×10⁷:1.

Clause 3. The process of Clause 1 or Clause 2, wherein R is a polymer having an Mₙ of about 100,000 g/mol or less.

Clause 4. The process of any one of Clauses 1-3, wherein R is an unsubstituted C₁-C₂₀ hydrocarbyl or a substituted C₁-C₂₀ hydrocarbyl.

Clause 5. The process of any one of Clauses 1-4, wherein the third precursor comprises mercaptosuccinic acid, 3-mercaptopropionic acid, mercaptopoly(ethylene glycol), or combinations thereof.

Clause 6. The process of any one of Clauses 1-5, wherein the Group 10 to Group 14 metal comprises Ni, Pd, Pt, Cu, Ag, Au, Cd, Hg, Zn, Al, Ga, In, Tl, Sn, Pb, or combinations thereof

Clause 7. The process of any one of Clauses 1-6, wherein the Group 10 to Group 14 metal comprises Au, Ag, Cu, Pt, Pd, Ni, or combinations thereof.

Clause 8. The process of any one of Clauses 1-7, wherein the amine is an alkylamine.

Clause 9. The process of Clause 8, wherein the alkylamine comprises tetradecylamine, oleylamine, octadecylamine, hexadecylamine, dodecylamine, or combinations thereof.

Clause 10. The process of Clause 8 or Clause 9, wherein the Group 10 to Group 14 metal comprises Au, Ag, Cu, or combinations thereof.

Clause 11. The process of any one of Clauses 1-10, wherein the functionalized metal is in the form of nanoparticles having an average particle size of about 2 nm to about 100 nm, as determined by transmission electron microscopy.

Clause 12. A composition, comprising:
a Group 10 to Group 14 metal; and
an organic group bonded to the Group 10 to Group 14 metal, the organic group comprising:

[symbol]

S—R—COOH,
wherein:
   [symbol] represents a bond to the Group 10 to Group 14 metal; and
   R is an unsubstituted hydrocarbyl, a substituted hydrocarbyl, an unsubstituted alkoxy, or a substituted alkoxy.

Clause 13. The composition of Clause 12, wherein R is a polymer.

Clause 14. The composition of Clause 12 or Clause 13, wherein R is an unsubstituted C₁-C₂₀ hydrocarbyl or a substituted C₁-C₂₀ hydrocarbyl.

Clause 15. The composition of any one of Clauses 11-14, wherein the organic group comprises

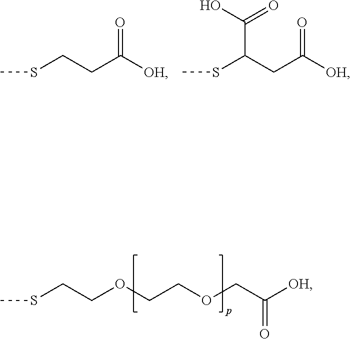

or combinations thereof, wherein:
p is a positive number of about 500 or less, and
[symbol] represents a bond to the Group 10 to Group 14 metal.

Clause 16. The composition of any one of Clauses 11-15, wherein p is from about 20 to about 200.

Clause 17. The composition of any one of Clauses 11-16, wherein the Group 10 to Group 14 metal comprises Ni, Pd, Pt, Cu, Ag, Au, Cd, Hg, Zn, Al, Ga, In, Tl, Sn, Pb, or combinations thereof.

Clause 18. The composition of any one of Clauses 11-17, wherein the Group 10 to

Group 14 metal comprises Au, Ag, Cu, Pt, Pd, Ni, or combinations thereof.

Clause 19. The composition of any one of Clauses 11-18, wherein:
at least a portion of the composition is in the form of nanoparticles; and
an average particle size of the nanoparticles is less than about 100 nm, as determined by transmission electron microscopy.

Clause 20. A device for detecting an analyte, comprising:
a substrate;
a source electrode and a drain electrode disposed on the substrate; and
a film disposed on a surface of the substrate, the film comprising:
   a Group 10 to Group 14 metal; and
   an organic group bonded to the Group 10 to Group 14 metal, the organic group comprising:

[symbol]

S—R—COOH,
wherein:
   [symbol] represents a bond to the Group 10 to Group 14 metal; and
   R is an unsubstituted hydrocarbyl, a substituted hydrocarbyl, an unsubstituted alkoxy, or a substituted alkoxy.

Clause 21. The device of Clause 20, wherein the film is further disposed on a surface of the source electrode, a surface of the drain electrode, or both.

Clause 22. The device of Clause 20 or Clause 21, wherein the device is configured to detect ammonia.

Clause 23. The device of any one of Clauses 20-22, wherein the film comprises a composition of any one of Clauses 12-19.

Clause 24. The device of any one of Clauses 20-23, wherein the device is configured to detect ammonia at a concentration of less than 500 ppm.

Clause 25. A device for detecting an analyte, comprising:
a substrate;
a source electrode and a drain electrode disposed on the substrate; and
a composition disposed on a surface of the substrate, the composition comprising a composition of any one of Clauses 12-19.

As used herein, and unless otherwise specified, the term “Cₙ” means hydrocarbon(s) having n carbon atom(s) per molecule, wherein n is a positive integer. The term “hydrocarbon” means a class of compounds containing hydrogen bound to carbon, and encompasses (i) saturated hydrocarbon compounds, (ii) unsaturated hydrocarbon compounds, and (iii) mixtures of hydrocarbon compounds (saturated and/or unsaturated), including mixtures of hydrocarbon compounds having different values of n. Likewise, a “Cₘ-C_{y}” group or compound refers to a group or compound comprising carbon atoms at a total number thereof in the range from m to y. Thus, a C₁-C₅₀ alkyl group refers to an alkyl group comprising carbon atoms at a total number thereof in the range from 1 to 50.

For purposes of this disclosure, and unless otherwise specified, the terms “hydrocarbyl radical,” “hydrocarbyl group,” or “hydrocarbyl” interchangeably refer to a group consisting of hydrogen and carbon atoms only. A hydrocarbyl group can be saturated or unsaturated, linear or branched, cyclic or acyclic, aromatic, or non-aromatic. For the purposes of this disclosure, and unless otherwise specified, the term “aryl” or “aryl group” interchangeably refers to a hydrocarbyl group comprising an aromatic ring structure therein.

For the purposes of this present disclosure, and unless otherwise specified, the terms “alkoxy” refers to an alkyl or aryl group bound to an oxygen atom, such as an alkyl ether or aryl ether group/radical connected to an oxygen atom and can include those where the alkyl/aryl group is a C₁-C₁₀ hydrocarbyl. The alkyl group may be straight chain, branched, or cyclic. The alkyl group may be saturated or unsaturated. Examples of suitable alkoxy can include methoxy, ethoxy, n-propoxy, iso-propoxy, n-butoxy, iso-butoxy, sec-butoxy, tert-butoxy, phenoxyl. “Alkoxy” includes ethers and polyethers.

Chemical moieties of the application can be substituted or unsubstituted unless otherwise specified. For purposes of this disclosure, and unless otherwise specified, a substituted hydrocarbyl, a substituted alkoxy, and a substituted aryl refers to a hydrocarbyl, alkoxy, and aryl radical, respectively, in which at least one hydrogen atom has been substituted with a heteroatom or heteroatom containing group, such as with at least one functional group, such as one or more of halogen (Cl, Br, I, F), NR*₂, OR* (e.g., OH or O₂H), SeR*, TeR*, PR*₂, AsR*₂, SbR*₂, SR*, SOₓ (where x=2 or 3), BR*₂, SiR*₃, GeR*₃, SnR*₃, PbR*₃, and the like or where at least one heteroatom has been inserted within the hydrocarbyl radical, such as one or more of halogen (Cl, Br, I, F), O, S, Se, Te, NR*, PR*, AsR*, SbR*, BR*, SiR*₂, GeR*₂, SnR*₂, PbR*₂, and the like, where R* is, independently, hydrogen or a hydrocarbyl.

Where isomers of a named molecule group exist (e.g., n-butanol, sec-butanol, iso-butanol, and tert-butanol), reference to one member of the group (e.g., n-butanol) shall expressly disclose all remaining isomers (e.g., sec-butanol, iso-butanol, and tert-butanol) unless specified to the contrary or the context clearly indicates otherwise. Likewise, reference to a compound without specifying a particular isomer (e.g., butanol) expressly discloses all isomers and stereoisomers (e.g., sec-butanol, iso-butanol, and tert-butanol) unless specified to the contrary or the context clearly indicates otherwise.

“Carboxyl group” and “carboxyl moiety” are used interchangeably herein. “Mercapto moiety” and “thiol moiety” and are used interchangeably herein.

As used herein, a “composition” can include component(s) of the composition and/or reaction product(s) of two or more components of the composition. Compositions of the present disclosure can be prepared by any suitable mixing process.

As is apparent from the foregoing general description and the specific aspects, while forms of the aspects have been illustrated and described, various modifications can be made without departing from the spirit and scope of the present disclosure. Accordingly, it is not intended that the present disclosure be limited thereby. Likewise, the term “comprising” is considered synonymous with the term “including.” Likewise whenever a composition, an element or a group of elements is preceded with the transitional phrase “comprising,” it is understood that we also contemplate the same composition or group of elements with transitional phrases “consisting essentially of,” “consisting of,” “selected from the group of consisting of,” or “Is” preceding the recitation of the composition, element, or elements and vice versa, e.g., the terms “comprising,” “consisting essentially of,” “consisting of” also include the product of the combinations of elements listed after the term.

For purposes of this present disclosure, and unless otherwise specified, all numerical values within the detailed description and the claims herein are modified by “about” or “approximately” the indicated value, and consider experimental error and variations that would be expected by a person having ordinary skill in the art. For the sake of brevity, only certain ranges are explicitly disclosed herein. However, ranges from any lower limit may be combined with any upper limit to recite a range not explicitly recited, as well as, ranges from any lower limit may be combined with any other lower limit to recite a range not explicitly recited, in the same way, ranges from any upper limit may be combined with any other upper limit to recite a range not explicitly recited. Additionally, within a range includes every point or individual value between its end points even though not explicitly recited. Thus, every point or individual value may serve as its own lower or upper limit combined with any other point or individual value or any other lower or upper limit, to recite a range not explicitly recited.

As used herein, the indefinite article “a” or “an” shall mean “at least one” unless specified to the contrary or the context clearly indicates otherwise. For example, aspects comprising “a metal” include aspects comprising one, two, or more metals, unless specified to the contrary or the context clearly indicates only one metal is included.

While the foregoing is directed to aspects of the present disclosure, other and further aspects of the disclosure may be devised without departing from the basic scope thereof, and the scope thereof is determined by the claims that follow.

What is claimed is:

1. A process for producing a functionalized metal, comprising:
introducing, under first conditions, a first precursor comprising a Group 10 to Group 14 metal with an amine to form a second precursor comprising the Group 10 to Group 14 metal; and
introducing, under second conditions, the second precursor with a third precursor to form the functionalized metal, the third precursor comprising an organic material having the formula:
HS—R—COOH,
wherein R is an unsubstituted hydrocarbyl, a substituted hydrocarbyl, an unsubstituted alkoxy, or a substituted alkoxy.

2. The process of claim 1, wherein the functionalized metal has the formula:
Mₘ(S—R—COOH)ₙ,
wherein:
M is the Group 10 to Group 14 metal; and
a ratio of m to n is from about 300:1 to about 1×10⁷:1.

3. The process of claim 1, wherein R is a polymer having an Mₙ of about 100,000 g/mol or less.

4. The process of claim 1, wherein R is an unsubstituted C₁-C₂₀ hydrocarbyl or a substituted C₁-C₂₀ hydrocarbyl.

5. The process of claim 1, wherein the third precursor comprises mercaptosuccinic acid, 3-mercaptopropionic acid, mercaptopoly(ethylene glycol), or combinations thereof

6. The process of claim 1, wherein the Group 10 to Group 14 metal comprises Au, Ag, Cu, Pt, Pd, Ni, or combinations thereof.

7. The process of claim 1, wherein the amine is an alkylamine.

8. The process of claim 7, wherein the alkylamine comprises tetradecylamine, oleylamine, octadecylamine, hexadecylamine, dodecylamine, or combinations thereof.

9. The process of claim 7, wherein the Group 10 to Group 14 metal comprises Au, Ag, Cu, or combinations thereof

10. The process of claim 1, wherein the functionalized metal is in the form of nanoparticles having an average particle size of about 2 nm to about 100 nm, as determined by transmission electron microscopy.

11. A composition, comprising:
a Group 10 to Group 14 metal; and
an organic group bonded to the Group 10 to Group 14 metal, the organic group comprising: [symbol]
S—R—COOH,
wherein: [symbol]
represents a bond to the Group 10 to Group 14 metal; and
R is an unsubstituted hydrocarbyl, a substituted hydrocarbyl, an unsubstituted alkoxy, or a substituted alkoxy.

12. The composition of claim 11, wherein R is a polymer.

13. The composition of claim 11, wherein R is an unsubstituted C₁-C₂₀ hydrocarbyl or a substituted C₁-C₂₀ hydrocarbyl.

14. The composition of claim 11, wherein the organic group comprises 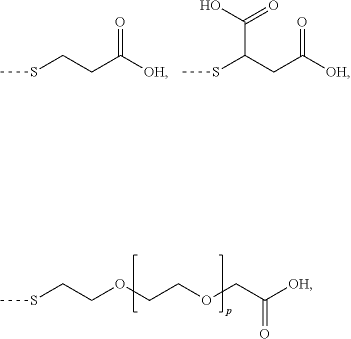
or combinations thereof, wherein:
p is a positive number of about 500 or less, and [symbol]
represents a bond to the Group 10 to Group 14 metal.

15. The composition of claim 14, wherein p is from about 20 to about 200.

16. The composition of claim 11, wherein the Group 10 to Group 14 metal comprises Au, Ag, Cu, Pt, Pd, Ni, or combinations thereof.

17. The composition of claim 11, wherein:
at least a portion of the composition is in the form of nanoparticles; and
an average particle size of the nanoparticles is less than about 100 nm, as determined by transmission electron microscopy.

18. A device for detecting an analyte, comprising:
a substrate;
a source electrode and a drain electrode disposed on the substrate; and
a film disposed on a surface of the substrate, the film comprising:
a Group 10 to Group 14 metal; and
an organic group bonded to the Group 10 to Group 14 metal, the organic group comprising: [symbol]
S—R—COOH,
wherein: [symbol]
represents a bond to the Group 10 to Group 14 metal; and
R is an unsubstituted hydrocarbyl, a substituted hydrocarbyl, an unsubstituted alkoxy, or a substituted alkoxy.

19. The device of claim 18, wherein the film is further disposed on a surface of the source electrode and a surface of the drain electrode.

20. The device of claim 18, wherein the device is configured to detect ammonia.
